(19) United States
(12) Patent Application Publication
(10) Pub. No.: US 2005/0124531 A1
(43) Pub. Date: Jun. 9, 2005

(54) Biological inhibitions induced by contact inhibitory factor

(75) Inventors: George Lipkin, New York, NY (US); Martin Jay Rosenberg, Brooklyn, NY (US)

Correspondence Address: DARBY & DARBY P.C., P. O. BOX 5257, NEW YORK, NY, 10150-5257, US

(73) Assignee: New York University, New YorK, NY (US)

(21) Appl. No.: 10/505,715

(22) PCT Filed: Feb. 24, 2003
(86) PCT No.: PCT/US03/05563

Related U.S. Application Data
(60) Provisional application No. 60/359,053, filed on Feb. 22, 2002.
(60) Provisional application No. 60/386,570, filed on Jun. 5, 2002.

(51) Int. Cl. A61K 38/18; A61K 31/7072; A61K 31/704; A61K 31/337

(52) U.S. Cl. 514/2; 514/27; 514/49; 514/34; 514/449

(57) ABSTRACT

The present invention is directed to a method for treating a patient suffering from inappropriate or excessive angio-genic activity comprising on administering to a patient in need of such treatment an amount of Contact Inhibitory Factor effective to treat said disease. Also disclosed herein are methods for preventing a cancer cell from metastasizing comprising administering to a patient in need of such treatment Contact Inhibitory Factor and a chemtherapeutic or immunotherapeutic agent. Also disclosed are pharmaceutical formulations for use in the methods.

This invention claims priority under 35 U.S.C. § 119(e) from: Provisional Application No. 60/359,053, filed Feb. 22, 2002 and Provisional Application No. 60/386,570 filed Jun. 5, 2002.

### FIELD OF THE INVENTION

This application is directed to the methods of inhibiting inappropriate or excessive angiogenesis or preventing cancer cells from metastasizing by administering Contact Inhibitory Factor.

### BACKGROUND OF THE INVENTION

Several lines of direct evidence now suggest that angiogenesis is essential for the growth and persistence of solid tumors and their metastases (Folkman, 1989; Hori et al., 1991; Kim et al., 1993; Millauer et al., 1994). To stimulate angiogenesis, tumors upregulate their production of a variety of angiogenic factors, including the fibroblast growth factors (FGF and BFGF) (Kandel et al., 1991) and vascular endothelial cell growth factor/vascular permeability factor (VEGF/VPF). However, many malignant tumors also generate inhibitors of angiogenesis, including angiostatin and thombospondin (Chen et al., 1995; Good et al., 1990; O'Reilly et al., 1994). It is postulated that the angiogenic phenotype is the result of a net balance between these positive and negative regulators of neovascularization (Good et al., 1990; O'Reilly et al., 1994; Parangi et al., 1996; Rastinejad et al., 1989). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993). The only known angiogensis inhibitor which specifically inhibits endothelial cell proliferation is angiostatin (O'Reilly et al. 1994).

A Contact Inhibitory Factor (CIF), derived from hamster (FF) and mouse (B16) cell lines has been shown to restore in vitro growth control to malignant melanoma cells, including contact-, serum-, and anchorage-dependent growth. The contact inhibitory effects are neither tissue nor species specific, extending to a broad spectrum of organ derived tumors, including colon, breast, brain, prostate and muscle. CIF also induces, in melanoma cells, the reappearance of pigment differentiation antigens, increases expression of Class I MHC antigens and enhances recognition and destruction of melanoma cells by cytotoxic T cells. CIF also reorganizes the cytoskeleton of melanoma cells in a more normal direction, decreases chemotaxis to laminin and decreases the surface expression of intercellular adhesion molecule 1 (ICAM-1) on melanoma cells.

CIF has been found to be non-toxic in vitro. In vivo it has been found to lead to regression of melanoma in hamsters (100%) and Lewis Lung carcinoma in mice (75%) without toxicity to the surrounding tissues.

Investigation of mechanisms which might have contributed to the regression of tumors demonstrated that CIF-mediated reversion of the malignant phenotype is accompanied by several changes in the antigenic profile of the melanoma cells. First, CIF induces the synthesis of vitiligo-related pigment differentiation antigens on mouse and hamster melanoma cells, which had lost these antigens (Lipkin et al., 1985), providing a potential target for immune destruction by both antibody-dependent cellular cytotoxicity and complement-mediated lysis (Norris et al., 1986). Secondly, CIF increases expression of Class I MHC antigens on mouse melanoma cells, with accompanying increase in susceptibility to lysis by cytotoxic (CD8) T lymphocytes. Both changes would make melanoma cells much better targets for the host's immune system.

However, an additional mechanism is suggested by the high vascularity of both melanomas and Lewis lung carcinomas. It is now well established that colonies of tumor cells require ingrowth of new blood vessels from the surrounding host vasculature in order to progress beyond a few mm in size (Folkman, 1985). Melanomas induce angiogenesis by secreting angiogenic molecules such as VEGF and FGF-2. Among melanocytic lesions there is a stepwise increase in vascularity with histologic progression from benign nevus to dysplastic nevus, primary cutaneous malignant melanoma and, finally, metastatic malignant melanoma (Barnhill et al., 1992). In fact, even for thin melanomas (<0.76 mm Breslow thickness), with a 5 year survival rate of 95%, high vessel counts are predictive of metastasis and death (Graham et al., 1994).

U.S. Pat. No. 4,307,082 issued Dec. 22, 1981 discloses a method for the extraction of CIF from media conditioned by the growth of a contact inhibited cell culture.

U.S. Pat. No. 4,530,784 issued Jul. 23, 1985 discloses a method for the large scale extraction of CIF. The protein component of the medium from the contact inhibited cell line was extracted by passage through a phenyl sepharose column. The extract was then lyophilized and resuspended before use.

Metastasis is caused by multiple genetic changes in evolving tumor cells, which confer on the affected cell the capability to progress through all of the steps in the metastatic cascade as set forth below.
1) Detachment of tumor cells from their primary site of growth.
2) Invasion of the surrounding tissues.
3) Penetration of blood and/or lymphatic vessel walls to enter into the circulation (intravasation).
4) Survival in the vasculature and dissemination to distant sites.
5) Attachment to distant capillary and post-capillary venular walls.
6) Penetration of the distant vessel walls to emerge in new organ sites (extravasation).
7) Establishment of new (secondary) tumor colonies.

Each of the above steps reflects a genetic change resulting in the acquisition of new structural, biochemical and enzymatic capabilities, which produce the metastatic cascade.

What is needed in the art are new uses for contact inhibitory factor which take advantage of the biological properties or this unique molecule.

### SUMMARY OF THE INVENTION

It has now been unexpectedly discovered that CIF inhibits the induction of angiogenesis by mouse melanoma cells by 2 different mechanisms: (1) it suppresses the secretion (export) of VEGF, a potent angiogenesis stimulus; and (2) it prevents the response of endothelial cells to another potent angiogenesis stimulus, bFGF.

It is expected that CIF will provide a more comprehensive therapy for a broad spectrum of cancers and other diseases where angiogenesis plays a significant role.

In one aspect the present invention provides a method for treating a patient suffering from inappropriate or excessive angiogenic activity comprising administering to a patient in need of such treatment an amount of Contact Inhibitory Factor effective to treat said patient.

In another aspect the present invention provides a method for inhibiting angiogenesis in a patient comprising administering to a patient in need of such treatment an amount of Contact Inhibitory Factor effective to inhibit angiogenesis in said patient.

In a further aspect the present invention provides a method for treating a disease of excessive or abnormal stimulation of endothelial cells comprising administering to a patient in need of such treatment an amount of Contact Inhibitory Factor effective to treat said disease.

The present inventors have also discovered that CIF inhibits metastasis of B16 mouse melanoma cells. This is an unusual and unexpected discovery because the present inventors do not know of any drug or agent which displays this activity.

It is expected that CIF will provide a more comprehensive therapy for a broad spectrum of cancers such as melanoma, colon carcinoma, brain tumors, mammary carcinoma and lung carcinoma.

In a further aspect, the present invention provides a method for preventing a cancer cell from metastisizing comprising contacting said cancer cell with an amount of CIF effective to prevent said cell from metastisizing.

In a still further aspect, the present invention provides a method for preventing a cancer cell from metastasizing comprising administering to a patient in need of such treatment an amount of
(a) Contact Inhibitory Factor; and
(b) a chemotherapeutic agent,
wherein the amounts of (a) and (b) in combination are effective to prevent said cancer cell from metastasizing.

In a still further aspect, the present invention provides a method for preventing a cancer cell from metastasizing in a patient who has received or is about to receive surgery to remove said cancer cell comprising administering
(a) Contact Inhibitory Factor; and
(b) an agent selected from an immunotherapeutic agent and a chemotherapeutic agent;
wherein the amounts of (a) and (b) in combination are effective to prevent said cancer cell from metastasizing.

In a still further aspect, the present invention provides a pharmaceutical formulation for preventing a cancer cell from metastasizing comprising
(a) Contact Inhibitory Factor; and
(b) an agent selected from a chemotherapeutic agent and an immuno suppressive agent;
wherein said amounts of (a) and (b) in combination are effective to inhibit said cancer cell from metastasizing, and a pharmaceutically acceptable carrier or diluent.

In a still further aspect, the present invention provides a pharmaceutical formulation for preventing a cancer cell from metastisizing comprising a) Contact Inhibitory Factor; and b) an immunotherapeutic agent; wherein the amounts of a) and b) in combination are effective to prevent said cancer cell from metastisizing. These and other aspects of the present invention will be apparent to those of ordinary skill in the art in light of the present description and appended claims.

### DETAILED DESCRIPTION OF THE INVENTION

### Definitions

“Angiogenesis” is defined herein as the process of new blood vessel formation (capillaries) from the pre-existing endothelial vasculature.

“Treatment” is defined as administration of CIF to a patient suffering from an ongoing illness, such as a carcinoma or administration to susceptible individuals to prevent an illness such as diabetic retinopathy or psoriasis.

“Treatment of tumors” is defined as preventing tumors from metastasizing.

“In conjunction with CIF” is defined as administration before, substantially simultaneously with or after administration of CIF.

The term about or approximately means within an acceptable error range for the particular value as determined by one of ordinary skill in the art, which will depend in part on how the value is measured or determined, i.e., the limitations of the measurement system. For example, about can mean within 1 or more than 1 standard deviations, per the practice in the art. Alternatively, about can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “purified” as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The phrase “pharmaceutically acceptable” refers to molecular entities and compositions that are generally regarded as safe, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

It has now been discovered that Contact Inhibitory Factor (CIF) inhibits inappropriate or excessive angiogenic activity. Therefore, CIF can be used to treat a wide variety of disorders in which angiogenesis is involved by administering an amount of CIF effective to treat said diseases (as defined below). Non-limiting examples of diseases which can be treated pursuant to the present invention include, but are not limited to, solid tumors; blood born tumors such as leukemias; tumor metastasis; benign tumors, for example hemangiomas, acoustic neuromas, neurofibromas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasis, rubeosis; Osler-Webber Syndrome; myocardial angiogenesis; plaque neovascularization; telangiectasia; hemophiliac joints; angiofibroma; and wound granulation. CIF, as is the case for other angiogenesis inhibitors, is useful in the treatment of disease of excessive or abnormal stimulation of endothelial cells such as occurs in atherosclerosis.

In light of the above finding, CIF can be used in a method for treating a patient suffering from a disease involving inappropriate or excessive angiogenic activity (as described above) by administering an amount of CIF effective to inhibit angiogenesis (as defined below).

CIF for use in the present invention can be prepared as described in U.S. Pat. No. 4,307,082 issued Dec. 22, 1981, U.S. Pat. No. 4,530,784 issued Jul. 23, 1985 or as described in Example 1 below.

An amount of CIF effective to inhibit angiogenesis and/or treat diseases of an inappropriate or excessive angiogenesis or excessive or abnormal stimulation of endothelial cells would broadly range between about 10 and about 50 anti-angiogenesis units per kg body weight of a recipient mammal. One anti-angiogenesis unit is defined herein as that amount of CIF which inhibits 50% of the growth of blood vessels induced by 50 ng basic fibroblast growth factor (bFGF) in the well known mouse corneal micropocket implant assay.

As shown below, the growth of bFGF-induced angiogenesis was inhibited by CIF in the Mouse Corneal Micropocket Implant Assay (as described in Kenynon, B. M. et al. 1996). This result suggests that the mechanism of action may involve the prevention of a response to bFGF by endothelial cells. In addition, the secretion of VEGF by melanoma cells was also reduced by 97% in cells treated with CIF.

Without wishing to be bound by theory, it is believed that there are two possible mechanisms by which CIF inhibits angiogenesis, either by (1) preventing the response of endothelial cells or (2) preventing the secretion of angiogenesis inducers by the tumor cells themselves. It is possible that both mechanisms could be in effect simultaneously.

It has now also been discovered that Contact Inhibitory Factor (CIF) inhibits metastasis of cancer cells. In light of this discovery, CIF can be used to treat mammals suffering from a cancer which has the potential to metastasize. Such mammals can be administered an amount of CIF, as described below effective to prevent the cancer cells from metastasizing.

In an alternative preferred embodiment, the present invention is directed toward the treatment of tumors (as defined above as preventing tumors from metastasizing), particularly solid tumors. Examples of solid tumors that can be treated according to the invention include sarcomas and carcinomas such as, but not limited to: fibrosarcoma, myxosarcoma, chondrosarcoma, osteogenic sarcoma, angiosarcoma, endotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, renal cell carcinoma, hepatoma, bile duct carcinoma, cervical cancer, testicular tumor, lung carcinoma, bladder carcinoma, epithelial carcinoma, melanoma, and retinoblastoma.

An amount effective to inhibit metastasis would broadly range between about 10 and about 50 anti-metastasis units of CIF per kg body weight of a recipient mammal.

One anti-metastasis unit of CIF is defined as that amount of CIF which inhibits metastasis by 50% in the chick embryo CAM model described below.

Pursuant to the present invention, CIF may be administered orally or preferably parenterally, e.g., intramuscularly, intraperitoneally and most preferably, intravenously.

Without wishing to be bound by theory, it is believed that CIF will be well-suited to be used in conjunction with other cancer treatments such as surgery, (which may involve inadvertently disseminating the tumor cells), chemotherapy, radiation and immunotherapy.

It is expected that CIF will not only prevent cancer cells from metastasizing but will also reduce the amount of chemotherapeutic agent administered by 2 to 10-fold, thereby reducing the side effects (e.g., nausea, leukopenia, hair loss, etc.) of chemotherapy.

Non-limiting examples of chemotherapeutic agents for use in the present invention include Taxol (Bristol-Myers Squibb, Princeton, N.J.) Adriamycin (Pharmacia & Upjohn, Peapack, N.J.), Etoposide (Baxter Healthcare Corp., New Providence, N.J.) and Gemcitabine (Eli-Lilly and Co., Indianapolis, Ind.)

Taxol is used for treatment of ovarian and breast cancer.

Standard Dose: 135-150 mg/m², i.v. over a 3 hour period for 3 weeks.

When administered in conjunction with CIF, the amount effective to treat the diesease: 50-100 mg/m², i.v., over a 3 hour period for 3 week.

Adriamycin (Doxorubicin) is used to treat colon cancer.

Standard Dose: 40-60 mg/m² i.v. for 21-28 days.

When used in conjunction with CIF the amount effective to treat the disease: 10-20 ml/m², i.v. for 21-28 days.

Etoposide is used for the treatment of Small cell lung cancer and Testicular Cancer

Testicular Cancer:

| | |
|---|---|
| Standard dosage: | IV infusion 50-100 mg/m²/day for 1-5 |
| | days, up to 100 mg/m²/day on days 1, |
| | 3, 5. Repeat at 3-4 week intervals. |
| When used in conjunction | 5-20 mg/m²/day for 1-5 days, |
| with CIF the amount effective | up to 20 mg/m²/day. |
| to treat the disease: | Same intervals as Standard dosage. |

Small cell lung cancer

| | |
|---|---|
| Standard dosage: | 35 mg/m²/day for 4 days, up to |
| | 50 mg/m²/day for 5 days |
| When used in conjunction with CIF the | 5-10 mg/m²/day Same intervals |
| amount effective to treat the disease: | as Standard dosage |
| Gemcitabine HCI is used for the | |
| treatment of Pancreatic Cancer | |
| Standard dosage: | IV infusion of 1000 mg/m²/day, |
| | once a day for 7 days, followed |
| | by one week's rest. Repeat cycle |
| | with 3 times a week, one week's |
| | rest |
| When used in conjunction with CIF the | 10-50 mg/m²/day. Same cycling |
| amount effective to treat the disease | schedule |

Immunotherapeutic agents for use ill the present invention include EDM 72000 (Merck & Co., Inc., West Point, Pa.)—a humanized monoclonal antibody to the EGF receptor (present in substantial quantities in>25% of breast cancers).

Standard Dose: 400-1600 mg i.v. in a one hour infusion, once weekly for 25 weeks or more.

When used in conjunction with 200-800 mg i.v., same intervals as standard CIF the amount effective to treat the disease: dose

The chemotherapeutic and immunotherapeutic agents may be administered before, after or substantially simultaneously with administration of CIF. Without wishing to be bound by theory, it is believed that the administration of CIF in this manner will:
a) diminish tumor cell heterogeneity thereby minimizing the emergence of resistant strains; and
b) reduce the dosage of the chemotherapeutic or immunotherapeutic agent. This reduction will result in decreased toxicity and a more prolonged and effective therapeutic regimen.

As shown below in Example 3, CIF treated mouse melanoma cells showed a highly significant decrease in their ability to metastasize in the well known chick embryo-chorioallantoic membrane assay. Those of ordinary skill in the art recognize that results obtained using this model system are predictive of efficacy in humans.

The CIF-containing formulations for use in the present invention include those suitable for oral, rectal, ophthalmic (including intravitreal or intracameral), nasal, topical (including buccal and sublingual), intrauterine, or parenteral (including subcutaneous, intraperitoneal, intramuscular, intravenous, intradermal, intracranial, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The present invention also provides pharmaceutical formulations comprising (a) CIF and (b) a chemotherapeutic or immunotherapeutic agent, wherein the amount of (a) and (b) in combination are effective to prevent a cancer cell from metastasizing. The formulation may be administered systemically, e.g., orally and are preferably administered parenterally, and most preferably intravenously. Formulations suitable for parenteral, administration may include aqueous and non-aqueous carrier and diluents such as sterile injection solutions, which may contain anti-oxidants, buffers, bacteriostatic agents and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

It should be realized that the pharmaceutical formulations of the present invention need not contain effective amounts of CIF or chemotherapeutic or immunotherapeutic agents as such effective amount can be obtained by administration of a plurality of such formulation

The present invention is described below in working examples which are intended to further describe the invention without limiting the scope thereof

### EXAMPLES

### Examples 1

In the Examples presented below, CIF was prepared as follows:

### Preparation of CIF-Containing Conditioned Medium

CIF-conditioned medium (CIF-CM) was obtained by treating confluent cultures of a contact inhibited cell line—the FF line—(available from The American Type Culture Collection, ATCC, Monassas, Va. as CCRL 1479) in Dulbecco's Minimal Essential Medium (DMEM GIBCO, Grand Island, N.Y.) in the absence of serum for 48 hours. The medium, now conditioned medium, then was collected. The CIF-CM was lyophilized and then redisolved in 1M NH₄HCO₃. It was then applied to a phenyl sepharose column which had been equilibrated with the same solution. The column was washed with 1M NH₄ HCO₃ until the non-binding fraction completely eluted; this material (Fraction I) was discarded. The column was then washed with dH₂O until the next fraction (Fraction II) completely eluted. The entire Fraction II was collected. Fraction II consists of the protein extract of the CIF-CM; CIF is bound to these proteins.

### Example 2

### CIF-Induced Anti-Angiogenic Activity

A. Materials

4-6 week old Mice (C57 Black) were obtained from Jackson Labs, Bar Harbor, Me.

Hydron pellets (Hydron Polymer Type NCC) were obtained from IFN Sciences, New Brunswick, N.J.).

Human Recombinant FGF-2 was obtained from R & D Systems, Minneapolis, Minn.

B. Methods

Hydron pellets were combined with sucrose ammonium sulfate BUKH, Meditec, Vaerlose, Denmark. The preparation and use of hydron pellets is described in Kenynon et al (supra).

Western Blotting

B16 cell-conditioned medium concentrated 80-fold by ultrafiltration in Centricon tubes (Amicon, Inc., Beverly, Mass., USA) was electrophoresed in SDS-12% polyacrylamide gel under reducing conditions and blotted to a polyvinylidene difluoride (PVDC) membrane (Immobilon P; Millipore, Bedford, Mass. USA) for 4 hours at 45V. The membrane was incubated with 5% skim milk (Carnation) in Tris buffer 10 mM, pH 7.4 overnight at 4° C. to block nonspecific binding. VEGF was detected by incubating the membrane with 0.2 μg/ml of antibody to human VEGF (alpha VEGF sc507; Santa Cruz Biotechnology, Santa Cruz, Calif. USA) for 1 hour at room temperature. Following incubation with horseradish peroxidase-conjugated donkey anti-rabbit IgG (1:5000) for 1 hour (Amersham Life Tecluiologies, Arlington Heights, Ill.), immune complexes were detected with the ECL™ detection system (Amersham, Life Technologies). The membranes were exposed to autoradiographic films (Hyperfilm M P; Amersham, Life Technologies) for 10 seconds to 1 minute. Scanning densitometry analysis of the Western blots was performed with Alpha Imager 2000 Documentation and Analysis System (Alpha Innotech Corporation, San Leandro, Calif.).

Results

Melanoma cells treated with CIF-containing conditioned medium in vitro reduced their secretion of VEGF by 97% as compared to cells treated with control medium.

In Vivo—The Mouse Corneal Micropocket Implant Assay

In order to evaluate the anti-angiogenic capacity of CIF, basic Fibroblast Growth Factor (bFGF), a known stimulator of angiogenesis, was implanted into the mouse cornea, either with or without CIF-CM as described in Kenyon et al (supra), and the resulting growth of blood vessels was measured.
1. 50 ng of basic Fibroblast Growth Factor (bFGF) was incorporated into each of 9 Hydron pellets.
2. 300 ng of the protein extract of CIF-CM was incorporated into each of 8 Hydron pellets.
3. 300 ng bFGF plus 50 ng of the protein extract of CIF-CM were incorporated together into each of 7 Hydron pellets.

Each pellet was inserted into a micropocket in the cornea of mouse, 7-9 in each of three groups. After 4 days, the extent of blood vessel ingrowth into the cornea from the limbus was evaluated in each mouse. The results are set forth in Table 1 below.

Results

TABLE 1

| Group | N | Avg. Area (mm²) |
|---|---|---|
| CIF-CM | 8 | 0.58 ± 0.38 |
| bFGF + CIF-CM | 7 | 1.35 ± 0.14 |
| bFGF | 9 | 3.48 ± 0.46 |

The presence of bFGF induced the growth of blood vessels, which occupied an average area of 3.48 mm². When CIF-CM was present together with the bFGF, the growth of blood vessels was significantly reduced; the average area was 1.35 mm², a 62% reduction in growth, according to the area occupied.

These results show that the CIF-CM exerted its anti-angiogenic effect by inhibiting the response to bFGF by endothelial cells.

In Vitro—The Inhibition of the Secretion of Vascular for Endothelial Growth Factor (VEGF) by CIF-CM.

Vascular Endothelial Growth Factor is a known stimulator of blood vessel growth into tumors. It is secreted by tumor cells and induces the growth of blood vessels from the host into the tumor, thus providing the blood supply for the tumor.

A known producer of VEGF, the murine B₁₆ melanoma cell line (available from the ATCC as ATCC CRL 6323) was grown in culture either in the presence or in the absence of CIF-CM. Both groups were initially grown in DMEM, 10% fetal bovine serum (FBS). When the flasks reached subconfluence, the medium was replaced with (1) the identical DMEM, 10% FBS (the controls), or (2) CIF-CM, 10% FBS. The cells were maintained at 37° and refed at 48 hours. At 72 hours, the media from each group was collected and analyzed for VEGF by inmunoprecipitation.

A 97% reduction was observed in the secretion of VEGF by the tumor cells treated with the CIF-CM.

### Example 3

### CIF-Induced Inhibition of Metastases

A. Materials

B16 Melanoma cells were obtained from the American Type Culture Collection (ATCC, Manassas, Va.) as ATCC CRL 6475.

Chick embryos were obtained from Charles River Spafas Co. (N. Franklin, Conn.)

B. Methods

### The Chick Chorioallantoic Membrane (CAM) Assay

B16 melanoma cells were seeded into 75 cm² cell culture flasks at subconfluent levels. After the cells had attached and spread, the medium in the experimental flasks was replaced with CIF-CM (15 mls per flask with 10% FBS); the control flasks received DMEM, 10% FBS. The cells were incubated for 24-48 hours, until the CIF-CM treated cells exhibited the expected morphological changes of phenotypic reversion such as parallel orientation of the cells, fibroblast-like morphology and contact inhibition of growth. Both groups of cells were then collected and injected into the veins of ten-day-old chick embryos (Chorioallantoic Membrane (CAM) Model), 300,000 cells per embryo (as described in Kim, J. et al., 1988).¹ After 7 days the lungs of each chick were examined and the number of metastases recorded. The data are shown in Table 1 below.
¹ These authors showed experimentally: (1) a close similarity between human and non-human tumor cells with respect tot their ability to adhere to and penetrate blood vessel walls in the CAM assay, and (2) that human tumor cells worked just as well as non-human cells in the CAM assay. Therefore, those of ordinary skill in the art recognize that this model is predictive of efficacy in humans.

C. Results In Vivo—The Chick Embryo Chorioallantoic Membrane Assay (CAM)

TABLE 1

| Treatment of | | |
|---|---|---|
| melanoma cells | N | Number of metastases |
| DMEM | 11 | 3.1 ± 3.0 |
| CIF-CM | 17 | 0.5 ± 0.9 |

The data showed that the melanoma cells treated with CIF-CM showed an 84% (p<0.003) reduction in the number of metastatic nodules.

CIF-CM treated mouse melanoma cells exhibited a highly significant decrease in their ability to metastasize in the chick embryo CAM model.

D. Chick Emboyo Intravasation Model

The experimental CAM model used above in Example 3, is an “extravasation” model, measuring the ability of potentially metastatic cells to emerge from blood vessels and to invade and colonize diverse tissues and organs. In addition, an “intravasation” model will be employed using the chick embryo. This model examines the ability of CIF to prevent potentially metastatic cells from penetrating into the blood vessels and entering into the circulation of the chick embryo. In this model, an inoculum of 300,000 tumor cells in 0.1 ml of culture medium (±CIF) is placed on the surface of the chorioallantoic membrane of a 10-day-old chick embryo, directly over a blood vessel. The egg is then incubated for one week at which time the organs are examined and scored for metastases. (modified from: Brooks, P. C., et al. [Montgomery, A. M. P. and to reference list and Cheresh, D. A. “Use of the 10-day-old Chick Embryo Model fro Studying Angiogenesis.” In: Methods in Molecular Biology. Vol. 129: Integrin Protocols. Ed. A. R. Howlett, Totowa, N.J., Humana Press.) Both the intravasation and extravasation models will be further investigated using various concentrations of CIF to determine a dose-response relationship. An additional metastatic model will be used in which potentially metastatic cells are injected into the tail vein of a mouse. After 7 days, the number of metastatic cells in the lungs are determined. One group of mice will receive daily injections of PBS (the control group) whereas the experimental group will receive daily injections of CIF in PBS. It is expected that the mice receiving the CIF will have fewer metastatic cells in the lungs of the treated animals.

References

Angiolillo et al., J. Exp. Med., 182:155-162 (1995).
Barnhill et al., Lab Investig. 67:331-337 (1992).
Brooks, P. C., et al., Methods in Molecular Biology. Vol. 129
Cao et al., J. Exp. Med., 182:2069-2077 (1995).
Chen et al., Cancer Res. 55:4230-4233 (1995).
Clapp et al., Endocrinology, 133:1292-1299 (1993).
Folkman, J., New York: Academic Press 43:175-203 (1985).
Folkman, J., Nature Med., 2:167-168 (1996).
Good et al., Proc. Nat. Acad. Sci. U.S.A., 87:6624-6628 (1990).
Graham et al., Am J Pathol 145:510-514 (1994).
Gupta et al., Proc. Natl. Acad. Sci U.S.A., 92:7799-7803 (1995).
Hori et al., Cancer Res., 51:6180-6184 (1991).
Kandel et al., Cell, 66:1095-1104 (1991).
Kenyon et al., Invest Opthlamol Vis Sci 37:1625-1632 (1996).
Kim, J. et al., Cell, 94:359-362, 1988)
Kim et al., Nature, 362:841-844 (1993).
Maione et al., Science, 247:77-79 (1990).
Millauer et al., Nature, 367:576-579 (1994).
Norris et al., J Invest Dermatol, 90:783, 789 (1988).
O'Reilly et al., Cell, 79:315-328 (1994).
Parangi et al., Proc. Natl. Acad. Sci. U.S.A., 93:2002-2007 (1996).
Rastinejad et al., Cell, 56:345-355 (1989).
Seghezzi et al., J Cell Biol, 141:1659-1673 (1998).
Singh et al., Proc Natl Acad Sci, 92:4562-4566 (USA) (1995).
Strieter et al., Biochem. Biophys. Res. Comm., 210:51-57 (1995).
Teske et al. Aust N Z J Opthalmol, 22:13-17 (1994).
Voest et al., J. Natl. Cancer Inst. 87:581-586 (1995).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for treating a patient suffering from inappropriate or excessive angiogenic activity comprising administering to a patient in need of such treatment an amount of Contact Inhibitory Factor effective to treat said disease.

2. The method of claim 1 wherein said disease is selected from solid tumors leukemias; tumor metastasis; hemangiomas, acoustic neuromas, neurofibromas, pyogenic granulomas; rheumatoid arthritis; psoriasis; ocular angiogenic diseases; diabetic retinopathy; retinopathy of prematurity; macular degeneration; corneal graft rejection; neovascular glaucoma; retrolental fibroplasia; rubeosis; Osler-Webber Syndrome; myocardial angiogenesis; plaque neovascularization; telangiectasia; hemophiliac joints; angiofibroma; and wound granulation.

3. The method of claim 1, further comprising administering a pharmaceutically acceptable carrier or diluent.

4. The method of claim 3, wherein said Contact Inhibitory Factor is administered systemically.

5. The method of claim 4, wherein said Contact Inhibitory Factor is administered parenterally.

6. The method of claim 5, wherein said Contact Inhibitory Factor is administered intravenously.

7. A method for inhibiting angiogenesis in a patient comprising administering to a patient in need of such treatment an amount of Contact Inhibitory Factor effective to inhibit angiogenesis in said patient.

8. The method of claim 7, further comprising administering a pharmaceutically acceptable carrier or diluent.

9. The method of claim 8, wherein said Contact Inhibitory Factor is administered systemically.

10. The method of claim 9, wherein said Contact Inhibitory Factor is administered parenterally.

11. A method for treating a disease of excessive or abnormal stimulation of endothelial cells in a patient comprising administrating to a patient in need of such treatment an amount of Contact Inhibitory Factor effective to treat said disease.

12. The method of claim 11 wherein said disease is atherosclerosis.

13. The method of claim 11, further comprising administering a pharmaceutically acceptable carrier or diluent.

14. The method of claim 13, wherein said Contact Inhibitory Factor is administered systemically.

15. The method of claim 14, wherein said Contact Inhibitory Factor is administered parenterally.

16. A method for preventing a cancer cell from metastisizing comprising contacting said cancer cell with an amount of CIF effective to prevent said cell from metastisizing.

17. The method of claim 16 wherein said cancer cell is a solid tumor.

18. The method of claim 17 wherein said solid tumor is selected from sarcomas and carcinomas.

19. The method of claim 18 wherein said sarcomas and said carcinomas are selected from fibrosarcoma, myxosarcoma, chondrosarcoma, osteogenic sarcoma, angiosarcoma, endotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, renal cell carcinoma, hepatoma, bile duct carcinoma, cervical cancer, testicular tumor, lung carcinoma, bladder carcinoma, epithelial carcinoma, melanoma, and retinoblastoma.

20. A method for preventing a cancer cell from metastasizing comprising administering to a patient in need of such treatment an amount of
(a) Contact Inhibitory Factor; and
(b) a chemotherapeutic agent,
wherein the amounts of (a) and (b) in combination are effective to prevent said cancer cell from metastasizing.

21. The method of claim 20 wherein said chemotherapeutic agent is selected from Taxol, Adriamycin, Etoposide and Gemcitabine.

22. A method for preventing a cancer cell from metastasizing in a patient who has received or is about to receive surgery to remove said cancer cell comprising administering to a patient in need of such treatment an amount of
(a) Contact Inhibitory Factor; and
(b) an agent selected from an immunotherapeutic agent and a chemotherapeutic agent;
wherein the amounts of (a) and (b) in combination are effective to prevent said cancer cell from metastasizing.

23. The method of claim 22 wherein said immunotherapeutic agent is EDM 72000.

24. The method of claim 23 wherein said chemotherapeutic agent or immunotherapeutic agent is administered before, after or substantially simultaneously with administration of said CIF.

25. The method of claim 24 wherein said chemotherapeutic agent is selected from Taxol, Adriamycin, Etoposide and Gemcitabine.

26. A pharmaceutical formulation for preventing a cancer cell from metastasizing comprising (a) Contact Inhibitory Factor; and
(b) a chemotherapeutic agent;
wherein said amounts of (a) and (b) in combination are effective to inhibit said cancer cell from metastasizing, and a pharmaceutically acceptable carrier or diluent.

27. The pharmaceutical formulation of claim 26 wherein said chemotherapeutic agent is selected from Taxol, Gemcitabine and Etoposide and Adriamycin.

28. A pharmaceutical formulation for preventing a cancer cell from metastisizing comprising
(a) Contact Inhibitory Factor; and
(b) an immunotherapeutic agent;
wherein the amounts of (a) and (b) in combination are effective to prevent said cancer cell from metastisizing.
